(19) United States
(12) Patent Application Publication
(10) Pub. No.: US 2023/0041626 A1
(43) Pub. Date: Feb. 9, 2023

(54) METHOD AND DEVICE FOR INTERMITTENTLY TRIGGERING A REFLEX-COORDINATED DEFECATION

(71) Applicant: Fred Göbel, Speyer (DE)

(72) Inventor: Fred Göbel, Speyer (DE)

(21) Appl. No.: 17/967,242

(22) Filed: Oct. 17, 2022

Related U.S. Application Data
(63) Continuation-in-part of application No. 16/026,669, filed on Jul. 3, 2018, now abandoned.
Application No. 16/026,669 is a division of application No. 14/239,991, filed on Jun. 13, 2014, now abandoned.

(30) Foreign Application Priority Data
Aug. 20, 2011 (DE) 10 2011 111 225.5
Nov. 21, 2011 (DE) 10 2011 118 943.6
Dec. 16, 2011 (DE) 10 2011 121 202.0
Mar. 22, 2012 (DE) 10 2012 005 607.9
Apr. 25, 2012 (DE) 10 2012 008 361.0

(51) Int. Cl. A61M 25/10; A61M 3/02; A61F 2/00; A61M 25/06; A61M 25/09

(52) U.S. Cl. CPC A61M 25/1002; A61M 25/10186; A61M 3/0295; A61F 2/0013; A61M 25/10185; A61M 25/1029; A61M 25/0662; A61M 2025/09116; A61M 25/10181; A61M 25/09041; A61M 2210/1064; A61M 25/10188; A61M 2025/1052; A61M 2025/105; A61M 2210/1067; A61M 3/0262; A61M 25/1009; A61M 2025/0687

(57) ABSTRACT

The invention relates to a method for filling an inflatable catheter balloon of a device for transanally introducing an infusion into the rectum or colon of a patient by means of a filling device, wherein a fill volume or a filling pressure prevailing in the catheter balloon is increased in two or more steps until there occurs a gradual, user-controllable, pneumatically initiated expansion of portions of the bowel wall, thus triggering a coordinated defecation reflex, wherein the timing and intensity of the triggering stimulus can be determined to the greatest possible extent by the user.

### REFERENCE TO PENDING PRIOR PATENT APPLICATIONS

This patent application is a continuation-in-part of pending prior U.S. patent application Ser. No. 16/026,669, filed 3 Jul. 2018 by Fred Gabel for METHOD FOR INTERMITTENTLY TRIGGERING A REFLEX-COORDINATED DEFECATION (Attorney's Docket No.: KUCH-59 DIV), which patent application is a division of prior U.S. patent application Ser. No. 14/239,991, filed Feb. 20, 2014 by Fred Gabel for TRANS-ANAL INFLOW CATHETER AND METHOD FOR INTERMITTENTLY TRIGGERING A REFLEX-COORDINATED DEFECATION (Attorney's Docket No. KUCH-59), which in turn is a 371 national stage entry of International (PCT) Patent Application No. PCT/EP2012/003535, filed Aug. 20, 2012 by Fred Gabel for TRANS-ANAL INFLOW CATHETER AND METHOD FOR INTERMITTENTLY TRIGGERING A REFLEX-COORDINATED DEFECATION, which patent application in turn: (i) claims benefit of prior German Patent Application No. 10 2011 111 225.5, filed Aug. 20, 2011; (ii) claims benefit of prior German Patent Application No. 10 2011 118 943.6, filed Nov. 21, 2011; (iii) claims benefit of prior German Patent Application No. 10 2011 121 202.0, filed 12/16/2011; (iv) claims benefit of prior German Patent Application No. 10 2012 005 607.9, filed Mar. 22, 2012; and (v) claims benefit of prior German Patent Application No. 10 2012 008 361.0, filed Apr. 25, 2012.

The eight (8) above-identified patent applications are hereby incorporated herein by reference.

### FIELD OF THE INVENTION

The invention relates to a method and a device for filling an inflatable catheter balloon of a device for transanally introducing an infusion into the rectum or colon of a patient by means of a filling device, more particularly to a method and a device for intermittently triggering a coordinated defecation reflex in the rectum or colon of a user with an individual anatomy of the rectum or colon and a current reflex status.

### BACKGROUND OF THE INVENTION

Catheters for transanally administering an infusion into the straight intestine (rectum) or large bowel (colon) of a patient have been known in a wide variety of embodiments for many years.

Inflow catheters can be implemented as simple tube elements, but for improved retention of the catheter in the rectum they are also provided with an intrarectally placed balloon element. Such balloon elements provide, in addition to an anorectally anchoring function, a certain sealing effect that retains in the bowel the fluid that has been introduced by the irrigation. The sealing capacity of inflow catheters fitted with an exclusively intrarectal balloon component is inadequate in many cases, however, and must be created by the user by continuously manually repositioning the surfaces of the sealing balloon on the floor of the rectum or manipulating the position of the catheter shaft atop the balloon.

A major risk associated with the use of ordinary commercial catheters that are inserted transanally in the bowel continues to be perforation of the bowel wall due to improper handling or the presence of previously injured or similarly weakened bowel wall structures. Such perforations of the intestine have been preventable heretofore only by suitable education and sensitization of the user with respect to this particular set of problems.

Over the past ten years, a novel infusion technology that goes beyond the use of conventional transanal infusions has become established; in this new approach, the reflex-coordinated, active evacuation of the rectum and large portions of the left colon adjoining the rectum is triggered by intermittent, relatively small-volume, intrarectally introduced infusions. By consistent voiding of these segments of the intestine, the patient can thus be brought to a state of so-called “pseudo-continence” regardless of existing incontinence problems. The so-called transanal irrigation (TAI) method can be performed by the patient himself.

The function of the rectally instilled infusion medium, apart from some degree of mobilization of stool, is primarily to moderately expand the lateral wall portions of the rectal ampulla (the terminal rectum), which then results in the triggering of the physiological stool evacuation reflex (reflex triggering). Whereas the rectally placed infusion medium usually causes such reflex triggering within a few minutes, it can take much longer, up to 30 minutes or more, for the administered irrigation volume to be fully voided from the rectum or colon. The relatively long time required for TAI limits its acceptance by many patients, and often rules this method out despite the fact that it is, as a rule, efficient for use by the individual.

Another problem is that many users are not able to manually bring irrigation catheters of conventional design into an adequately sealing position and keep them there.

Furthermore, as the balloon component of a conventional catheter is expanded inside the rectum, a reflex-triggering expansion of portions of the bowel wall can occur when the filling process has only just begun, thereby leading, inter alia, to a reflex-like opening of the anus and thus sometimes causing the anchoring balloon to slip out of the rectum.

Another disadvantage of such a prematurely triggered reflex, which shall be called a pathological reflex, against a coordinated physiological reflex is that the latter begins at the upper arch of the descending colon and then transports the intestinal contents from there downwards to the sigmoid colon and from there to the rectum, from where it is finally drained through the anus. After such a coordinated physiological reflex, the descending colon, sigmoid colon and rectum is entirely empty, and the user is pseudo-continent for the following hours. Compared to this, a pathological reflex is not coordinated and therefore does not transport the intestinal contents towards the rectum and anus, and as a consequence, the the user is not pseudo-continent afterwards.

Furthermore, commercially available devices for intermittent transanal irrigation, such as, for example, Peristeen Anal Irrigation, made by Coloplast, Denmark, are of relatively elaborate design, and are often only conditionally suitable for use outside the patient's familiar domestic surroundings.

### SUMMARY OF THE INVENTION

To improve user acceptance of transanal irrigation, a catheter technique would be desirable which, once the catheter shaft is inserted in the rectum, holds it in its transanal position without the need for continuous repositioning, and both keeps the body of the shaft from sliding on into the bowel, with potentially traumatic consequences, and reliably prevents the catheter from accidentally slipping out of the anus prematurely.

Greater convenience in terms of the transanal placement of the catheter is offered in this regard by inflow catheters having a double balloon arrangement. Here, two separate balloon elements, which are usually filled simultaneously via a single filling lumen, are mounted in spaced relation on the catheter shaft. These come to lie on both sides of the anal canal, i.e. the internal and the external, thus mutually taking on the structures of the anus. When the wall of the balloon is expanded under pressure, the usually elastic implementation of the balloon material brings about a spherical enlargement of the balloon bodies and thus a certain axially exerted squeezing of the anus disposed between the balloon bodies. The dislocation-proof positioning of the catheter shaft obtained with such catheters can be assumed to be relatively reliable. A problem resides in the sometimes high filling pressures needed for the elastic expansion of the envelope, which are felt by patients as an uncomfortable or even painful foreign body sensation. Further, the elastic expansion of the intrarectal balloon to a tightly filled sphere can lead to direct triggering of the defecation reflex, which, in turn, in the presence of simultaneous rectal contraction and declining sphincter muscle tone, will in the most unfavorable case cause the catheter to slide out and the irrigation fluid to be voided prematurely.

DE 10 2004 033 425 B4 describes a sealing system for the management of rectal or anal incontinence having a particular embodiment for the tamponade of bleeding hemorrhoids, presenting a waisted balloon element having a terminal intrarectal or preanal section. The balloon envelope of the device is placed on the balloon-supporting shaft body in such a way that during the filling process, the intrarectal balloon segment moves in the direction of the rectal floor and there exerts a tamponade pressure on the bleeding venous blood vessels. The preanal balloon segment concurrently moves toward the external anal opening. The result is a tamponade effect oriented axially to the anus from both sides. In addition to the axial tam ponade of the bleeding, the middle, waisted section of the balloon expands outward radially to the wall of the anal canal. It is preferably shaped with a diameter that exceeds the diameter of the opened anal canal. In the described device for the acute management of venous anorectal bleeding, in the in-use state the tip of the shaft body protrudes freely and unprotectedly into the intestinal lumen and poses a potential risk of injury.

WO 2007/118621 A1 describes a similar dumbbell- or hourglass-shaped balloon sealing system. The object there is to provide a seal against involuntary leakage of stool for intermittent periods of a few hours in chronically anorectally incontinent patients. This managing device also prominently features the rolling movement of the terminal balloon segments from both sides axially toward the anus. Here again, in the transanally placed state, the tip of the shaft body supporting the balloon protrudes freely and potentially traumatizingly into the intestinal lumen.

Both devices describe an hourglass- or dumbbell-shaped balloon configuration, the anus being received in the waisted region of the balloon, i.e. the region that is tapered relative to the terminal portions thereof. Such shaping of the balloon not only results in relatively good securing of the catheter against dislocation, due to the sealing of the balloon inside the anal canal on all sides, but also makes for a considerable improvement in sealing performance over that of conventional double balloons. In addition, both devices make it possible to largely avoid any expansion of the balloon envelope that could trigger irritation, pain or a defecation reflex, since the balloon wall is preferably already shaped to a reference dimension or beyond. Thus, the filling or unfolding of the balloon to its reference state requires only a low filling pressure, equal to or only slightly greater than the pressure prevailing in the rectum or abdomen at the time.

Nevertheless, a particular disadvantage of the embodiments described in DE 10 2004 033 425 B4 and WO 2007/118621 A1 has proven to be that the distal tip of the shaft body supporting the balloon becomes freely exposed during the filling process and protrudes into the intestinal lumen, potentially causing irritation or injury to the bowel wall there during use.

The same problem occurs at the closure system for managing rectal or anal incontinence disclosed in the US 2007/0 123 661 A1. At this document, even the distal-most balloon is fixed to the catheter shaft in a rather big distance from the distal tip of the catheter shaft, so that—if the distal-most balloon is not urged towards the distal tip of the catheter shaft, this tip is not protected by the catheter balloon from injuring the intestinal mucosa. Furthermore, all balloons which are disclosed in this document and are intended to be placed inside of the rectum or colon of a user are designed with a rather longitudinal shape, where the distance from the radial-most extension of the balloon to the lateral surface of the catheter shaft is smaller than the axial dimension of the balloon.

Therefore, such a balloon is not capable of producing a force onto the lateral colon wall of the user, but develops upon inflation in an axial direction from the rectum towards the colon of the user.

The arrangement disclosed in the WO 2008/103 788 A1 suffers from the same disadvantage. This document describes a seal for a rectal or ostomy appliance. At this document, the envelope of the catheter balloon is fixed at three fixation points, namely (i) in the area of the rectum, the distal end of the tubular balloon envelope is fixed to the catheter shaft itself, (ii) in the preanal region, the the proximal end of the balloon envelope is fixed to at a flexible rear wall of the device, and (iii) at an area between these two fixation points, an intermediate portion of the balloon envelope is fixed for a third time, namely to the catheter shaft in the area of the anus. Such a third fixation point reduces the capability of the balloon to move freely along the catheter shaft in order to center and seal the balloon in the area of the anus or rectum. But what is much worse, the balloon balloon is not capable of producing a force onto the lateral colon wall of the user, but develops upon inflation in an axial direction from the rectum towards the colon of the user. Finally, this balloon is made of an elastic material and therefore does not show any wrinkles as it is placed inside the rectum, even if it is not inflated, when it is rather small and contracts towards the catheter shaft, as this balloon is not preshaped to a reference volume corresponding to the rectum or colon of a user.

The document UA 68 194 A refers to a method for treating inertness of the rectum. The former arrangement known from this document does neither comprise a catheter nor a tubular or annular balloon fixed to such catheter at two fixation points. Instead, this document discloses a first ellipsoid balloon for placement in the rectum and a second balloon for placement in the anal canal. Both balloons are spaced apart from each other for about 2 cm. The rectal balloon is made of elastic thin latex rubber, which is a rather compliant material and can be blown up to an unlimited volume, thereby potentially inducing serious injuries to the user. Therefore, such equipment cannot be used by the user himself, but only by skilled personnel. Furthermore, this method is not used for triggering a coordinated physiological reflex in order to empty the descending colon, the sigmoid colon and the rectum, but it is used only for training of the contractile activity of the colon. For this purpose, the pressure in the balloon is varied with a frequency of 3 to 6 times per minute. So, a single pressure pulse is only 5 seconds in case of a frequency of 6 times per minute for a whole pressure cycle, up to 10 seconds in case of a frequency of 3 times per minute for a whole pressure cycle. Even if a defecation reflex would be triggered by such a short pressure pulse, it would not lead to a coordinated physiological reflex which would be able to empty the descending colon, the sigmoid colon and the rectum. Instead, such short pulses would only lead to the aformentioned pathological reflexes which are not able to clean the descending colon, the sigmoid colon and the rectum. Moreover, the rectal balloon has a ellipsoidal shape, but no discoidal shape. Therefore, the influence on the lateral walls of the rectum is weakened as the balloon “escapes” the rectum in the axial direction towards the colon, especially due to a missing catheter shaft which would otherwise define a constant length of the balloon and promote a more radially expansion of the balloon in case of its inflation.

Finally, the WO 2009/144 028 A1 discloses a device for a drainage of stool from the colon of a patient. There, a catheter shaft is missing, too, and therefore, the balloon will attain a more axial shape than a discoidal shape upon inflation, and for this reason, the influence on the lateral walls of the rectum is rather weak.

Another disadvantage of such a prematurely triggered reflex, which shall be called a pathological reflex, against a coordinated physiological reflex is that the latter begins at the upper arch of the descending colon and then transports the intestinal contents from there downwards to the sigmoid colon and from there to the rectum, from where it is finally drained through the anus. After such a coordinated physiological reflex, the descending colon, sigmoid colon and rectum is entirely empty, and the user is pseudo-continent for the following hours. Compared to this, a pathological reflex is not coordinated and therefore does not transport the intestinal contents towards the rectum and anus, and as a consequence, the the user is not pseudo-continent afterwards.

The problem initiating the invention is to eliminate these disadvantages of the known prior art.

This problem is solved, in an arrangement of the cited species, by virtue of the fact that a catheter shaft is provided as well as a catheter balloon in the form of an endless cut, that is an annual or tubular cut with only two circularly extending edges which afterwards form ends tapering to the shaft dimension of the catheter shaft supporting the balloon, and which are fixed on the preferably outer jacket surface of the catheter shaft, preferably in a simply invaginated or inverted manner.

By doing this, a catheter is provided, the catheter comprising:
a catheter shaft, and
a catheter balloon surrounding the catheter shaft and being fixed to it, wherein the catheter balloon comprises a balloon envelope:
which has a tubular or an annular geometry with two ends which are fixed to the catheter shaft at two fixation points in a fluid-tight manner in order to encompass a fluid-tight fillable compartment, wherein the distal fixation point of the catheter balloon is near the distal end of the catheter shaft, and
which surrounds the catheter shaft between the two ends of the balloon in a freemoving manner, as the balloon envelope is not attached or fixed to the catheter shaft at any third location between the two fixation points of the ends of the balloon envelope at the catheter shaft, and
which, during manufacturing of the catheter balloon, is preshaped to a reference dimension V_{REF} of a balloon filling volume V_{BF} as a maximum filling volume of the compartment in the balloon, which is achievable by inflation, but without any stretching of the balloon envelope, and which reference dimension V_{REF} of the balloon filling volume V_{BF} corresponds to a dimension of the rectum or colon of the user, and
which is manufactured from a material which, although being flexible, is non-stretchable or only slightly stretchable so that, at or above the reference dimension V_{REF}, the ballon filling volume V_{BF} of the catheter balloon instantly assumes or asymptotically approximates an unvarying dimension limit V_{UDL} at a pressure in the range of 10 to 120 mbar applied to the interior of the balloon:

V_{UDL}>V_{REF}.

Two fixation pointes on a catheter shaft, the distal one thereof near the distal tip of the catheter shaft can provide the balloon with a rather stable axial dimension, so that it is able to expand primarily in a radial direction. This feature can be promoted, if the wall thickness in the area near the ends of the balloon is greater than in an intermediate area between both fixation points, or if both flanks of the balloon extend from one of both fixation points towards a more edge-like periphery without a cylindrical section between both flanks of the balloon, so that the inflated balloon adopts a discoidal shape with a peripheral edge surrounding the catheter shaft in a maximum distance.

With such a device being provided, the following steps can be carried out.

In a first step, inserting the catheter in the rectum or colon of the user and providing an initial fill volume V_{IF,1} or an initial filling pressure p_{IF,1} of air or another fluid in the catheter balloon in order to inflate the catheter balloon below the reference dimension V_{REF} of the balloon filling volume V_{BF} thereof in a pressure-neutral manner, i.e. wherein the initial filling pressure p_{IF,1} inside the catheter balloon is equal to a physiologically acting intra-rectal acting pressure p_{IR} that is prevailing in the rectum of the user, producing a sealing and anchoring effect without any stretching of the envelope of the catheter balloon and causing neither a trans-mural pressure p_{TM,1} beyond the physiologically intra-rectally acting pressure p_{IF} nor a reflex triggering expansion of portions of the lateral rectal wall exposed to the catheter balloon, according to the following formulas:

p_{IF,1}=p_{TM,1}=p_{IR},

and wherein the initial fill volume V_{IF,1} of the catheter balloon is below the reference dimension of the balloon filling volume V_{REF}:

V_{IF,1}<V_{REF},

so that the envelope of the balloon is not stretched; and in a second step, increasing such initial fill volume V_{IF,1} or such initial filling pressure p_{IF,1} to an increased fill volume p_{IF,2} or an increased filling pressure p_{IF,2} by pumping, squeezing or injecting air or another fluid into the catheter balloon by the user until there occurs a gradual, user-controllable expansion of portions of the lateral rectal wall exposed to the catheter balloon in such a way that the degree of filling of the balloon is best adapted to the individual anatomy, allowing for an even distribution of the trans-mural pressure p_{TM,2} exerted by the balloon onto the exposed rectal structures, gradually increasing from the physiologically acting intra-rectal pressure p_{IR} to a force level suited for triggering a coordinated physiological defecation reflex of the individual user, according to the following formula:

p_{IF,2}=p_{TM,2}>p_{IR},

and wherein the increased fill volume V_{IF,2} of the catheter balloon is still below the reference dimension of the balloon filling volume V_{REF}:

V_{IF,2}<V_{REF},

so that the envelope of the balloon is not stretched.

Wheras the first step only serves the purposes to center the balloon in the rectum and to seal the balloon in or against the anus, in the second step, the pressure is raised a little, in order to generate a moderate trans-mural pressure slightly beyond the intra-rectal pressure which does not trigger a reflex at once. In stead, such a pressure level slightly raised above the intra-rectal pressure is kept constant for about 1 minute to several minutes, typically for 1 minute to 5 minutes, or for 2 minutes to 5 minutes, and within this waiting time, the rectum slowly establishes a coordinated physiological reflex, which is able to clear the descending colon, the sigmoid colon and the rectum from the intestinal contents. There shall be no fluctuations within this waiting time of about 1 minute to 5 minutes. Instead, the constant pressure level shall simulate a gradual increasing of the filling of the rectal ampoule in order to trigger a coordinated physiological reflex with a sufficient force to empty the descending colon, the sigmoid colon and the rectum.

The different pressure levels are defined as follows:

The balloon filling pressure p_{BF} is the pressure inside of the catheter balloon which can be influenced by pumping air or another fluid into the balloon or by emptying the balloon gradually by opening of a valve, and which can be measured by a manometer which can be integrated with a pump. It should be noted, that the pressure p_{BF} is also influenced by shifting air or another fluid into the balloon with a syringe. The only difference of these two methods is that by a pump, the pressure is directly controlled, whereas by use of a syringe, a predefined volume of air or liquid corresponding to the fill volume of the syringe is shifted into the balloon. Therefore, the first method is addressed as a pressure-controlled filling method, whereas the second method is addressed as a volume-controlled filling method, also bot filling methods affect both the filling pressure p_{BF} as well as the fill volume V_{BF} of the balloon.

While the balloon filling pressure p_{BF} is the pressure at the inner surface of the balloon envelope, the trans-mural pressure p_{TM} is the pressure at the outer surface of the balloon envelope. If the balloon envelope is not stretched, both pressures are equal to each other:

p_{BF}=p_{TM}.

On the other hand, if the balloon envelope indeed is stretched, the trans-mural pressure p_{TM} is not equal to the balloon filling pressure p_{BF} anymore, but is reduced by the tensioning force in the balloon envelope. Then, the relation is:

p_{BF}>p_{TM},

although the trans-mural pressure p_{TM} increases, too, if the the balloon filling pressure p_{BF} is raised.

Furthermore, the intra-rectal pressure p_{IR} is the physiological pressure exerted in the rectum by the body or, respectively, anatomy of the user, in a similar way as the intra-abdominal pressure inside the abdomen of the user. This pressure is not affected, neither by the trans-mural pressure p_{TM} nor by the balloon filling pressure p_{BF}.

As the envelope of the catheter balloon is not completely unfolded to the reference dimension V_{REF} of the balloon filling volume, neither in the first step nor in the second step, it is wrinkled in all these steps. Only in case of a maloperation, that is if the user inflates the balloon beyond its preshaped reference dimension V_{REF}, the wrinkles disappear at the envelope. In order to prevent injuries of the user in case of such a malopertion, the envelope is made of a material which achieves a rather unvarying dimension V_{UDL}, where any further expansion comes to a stop.

In many cases, it has proven to be advantageous, if the triggering effect can be increased by filling a liquid irrigation inflow fluid directly into the rectum of the user via a central lumen of the catheter shaft, which is open at both ends of the catheter shaft. Preferably, this can be done between the first and the second step of the method according to the invention. The additionally poured in liquid irrigation inflow fluid assists the fill volume inside the balloon in triggering a coordinated physiological emptying reflex.

To prevent, insofar as possible, lesions due to the tip of the shaft, the invention describes a specific, particularly advantageous ratio of the length of the middle, waist-like balloon segment to the invagination (inversions), on both sides, of the fixation points of the ends of the balloon shaft to the catheter shaft. This ratio ensures that in the filled, transanally positioned state, the tip of the catheter shaft spontaneously retracts into the intrarectal balloon segment and is nestled atraumatically there. The atraumatic securing of the catheter tip inside the intrarectal balloon segment is also ensured according to the invention when the shaft axis undergoes the deflections inside the anal canal that typically occur during use, of the kind readily caused by a tug or push on the tube connection through which the catheter is guided in.

The invention further addresses the problem of preventing undesired or premature triggering effects due to the filled balloon as it unfolds in the rectum. Triggering of the defecation reflex can be prevented in most cases by preshaping the balloon to the reference dimension or beyond (residual dimensioning), since the filling pressures necessary for anchoring and sealing the catheter are largely the same as, or need be only a few millibars higher than, the pressures prevailing in the rectum or in the abdomen. The atraumatic securing of the tip of the catheter in the region of the intrarectal balloon segment is ensured even at such low filling pressures of, for example, 10 to 25 mbar.

Shaping the balloon to its reference dimension further makes it possible for the user, by increasing the filling pressure or fill volume of the balloon from the initial filled state in which the balloon primarily has a sealing and anchoring effect, to induce a well-controllable and gradually increasable expansion of the bowel wall adjacent the catheter balloon, ultimately leading to controlled triggering of the defecation reflex with individually adjusted intensity.

In addition to the initial expansion of the wall of the rectum, a further suitable increase in the filling pressure in the dumbbell-shaped, waisted catheter balloon additionally causes a dilation of the anus or the anal sphincter, constituting another efficient trigger stimulus.

The intensity of such pneumatic expansion of the bowel wall or the anus can substantially exceed the intensity of reflex-triggering expansion with fluid media. Due to the more intense stimulus, several sequentially occurring cycles of evacuation reflexes can be triggered even though the stimulation is given only once.

This pneumatic expansion stimulus that can be well controlled by the user can also reduce the necessary amount of infusion fluid in many cases, which in turn can considerably shorten the time needed to evacuate the infusion from the rectum, thereby ultimately opening up the method to many users as a therapeutic option for self-administration.

The optional combined pneumatic/liquid triggering of reflexes described here eliminates the need for elaborate technical implementation of irrigation appliances, since in the preferred use case the necessary irrigation volume can be kept so small that it can be connected directly, as a compact, ready-to-use solution, via a fixed feed conduit to an inflow catheter designed according to the invention, and thus, in an ideal manner, be used as a disposable product.

For this purpose, the irrigation solution is preferably filled into a cylindrical, bag-like container, which the user can conveniently introduce intrarectally by squeezing it out with the hand.

The inflow catheter according to the invention further comprises a preferably fixedly glued-in filling conduit for charging the catheter balloon with filling pressure. The unit for filling the balloon is preferably implemented in reusable form, and can be configured as a hand-operated pump balloon connectable via a coupling, or alternatively a pumped balloon with a pressure-indicating manometer. Alternatively, volume-controlled filling of the balloon is also conceivable. Regardless of the filling arrangement, the user can determine by stepwise approximation the degree of filling of the balloon that is optimal for him, that which is best adapted to his individual anatomy and his current reflex status.

To ensure the most practicable catheter insertion possible and the reliable transanal placement of the tapered portion of the dumbbell-shaped balloon element, the catheter shaft is preferably equipped in the proximal, preanal region with gripping depressions to accommodate the fingers gripping the catheter during insertion. If the user has no sensitivity in the pelvic or anal region, he guides the catheter in with his fingers until they abut the anus, and can thus avoid uncontrolledly deep insertion.

### BRIEF DESCRIPTION OF THE DRAWINGS

Further features, characteristics, advantages and effects of the invention will become apparent from the following description of preferred embodiments of the invention and by reference to the drawing. Therein:

FIG. 1a shows an embodiment of a catheter according to the invention in a schematic longitudinal section, the balloon ends being mounted on the catheter shaft in an inverted manner according to the invention;

FIG. 1b shows the geometric structure of the apex of the distal balloon radius;

FIG. 1c shows the balloon body described in FIG. 1a in a freely unfolded state under low filling pressure, outside the anus;

FIG. 1d shows the balloon body described in FIG. 1a in a transanally placed state, under filling pressure;

FIG. 1e shows the geometric structure of the distal plane tangent to the intrarectal balloon section;

FIG. 2 is a representation similar to FIG. 1a of another embodiment of the invention, in which a catheter tip extends beyond the forward fixation line of the end of the balloon shaft;

FIG. 3 is a representation similar to FIG. 2 showing additional auxiliary lines, such that the reference point for determining the inversion depth B is not the forward balloon radius, but rather the largest diameter D of the intrarectal balloon segment;

FIG. 4 shows a shaped balloon envelope in the unfilled state, provided in a particularly advantageous manner for rectal insertion and reliable transanal placement and unfolding of the balloon;

FIG. 5 shows a modified embodiment of the invention with a catheter shaft waisted in its transanal section,

FIG. 6 shows a manually operable pump manometer with a pressure scale equipped for multi-stage, sequential filling of the catheter balloon,

FIG. 7 shows a further modified embodiment of the invention with an infusion container fixedly connected to the catheter shaft and a filling tube for charging the catheter balloon with filling pressure.

### DETAILED DESCRIPTION OF THE PREFERRED EMBODIMENTS

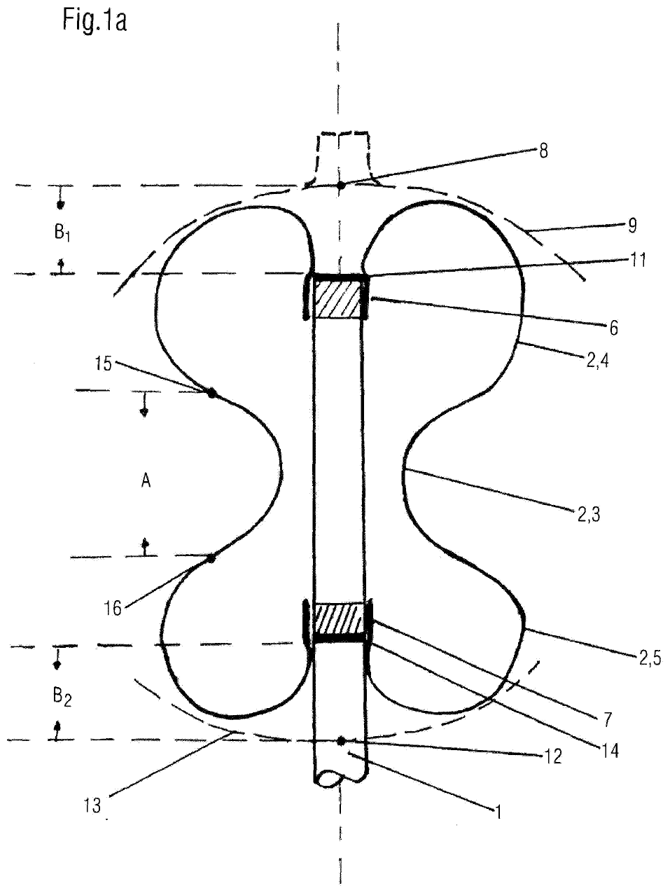

FIG. 1a shows a longitudinal section through an inflow catheter with a dumbbell- or hourglass-shaped balloon element 2 disposed at the distal end of the catheter shaft 1. The balloon element has, for example, a spherical or discoid expansion at each end. Disposed in the middle region between the terminal enlargements is a cylindrically or approximately cylindrically shaped segment 3 of reduced diameter, which continuously connects the intrarectal enlargement 4 to the preanal enlargement 5.

The balloon 2 terminally comprises two balloon shaft ends (6, 7) for attaching the balloon to the shaft. During the mounting of the balloon, the shaft ends 6 and 7 are invaginated (inverted) into the inside of the balloon by a defined magnitude B and in this position, in which they are displaced toward each other, are fixed on the shaft 1, for example by gluing or welding.

The sum of the magnitudes B of the inversions at both ends should be at least equal to the length of the tapered intermediate piece A (A smaller than/equal to the sum of the magnitudes B).

The inversion depth B at the end of the balloon facing the patient or the rectum corresponds to the distance from the apex 8 of the distal, intrarectal balloon radius 9 to the distal fixation line 11 of the balloon end 6 on the catheter shaft.

The distal radius 9 corresponds to the frontal radius during free, non-inverted unfolding of the completely filled but not pressurized balloon (broken line). An exemplary rule for geometrically determining the apex 8 to good approximation is illustrated in FIG. 1b. In particular, the drawing shows the two inflection points WP of the longitudinal section through the distal balloon end, which, by the inversion of the distal region of the balloon, result in the spherical or discoid or approximately semitoroidal region of the intrarectal section of the balloon. The normals 9e, 9f to the tangent to the longitudinal section of the balloon intersect with the longitudinal axis X of the catheter shaft at point M, and a circle K around this point M at the distance M-WP yields the forward apex 8, which marks the distal limit for the catheter shaft in its neutral, non-deflected initial position.

It can be seen in FIG. 1e that a straight line can also be passed through points WP; this straight line represents the plane Z which after invagination of the forward end of the balloon is now tangent distally to the intrarectal portion of the balloon, and can also be taken as a measure of the distalmost position of the catheter shaft in its neutral, non-deflected initial position.

On the side of the balloon facing away from the patient, the inversion depth B corresponds to the distance from the apex 12 of the proximal, preanal balloon radius 13 to the proximal fixation line 14 of the balloon end 7 on the catheter shaft.

Radius 13 corresponds to the proximal radius when the balloon is freely unfolded without inversion. The geometric determination of apex 12 is similar to the approximation described in FIG. 1b.

The length of intermediate piece A is determined by determining the distance between the transitions of the shoulder radii 15 and 16 (inflection points) of the mutually facing shoulder surfaces of balloon segments 4 and 5.

The inversion depths, lengths and distances are each determined in the filled state under filling pressure, the filling pressure being so selected that the balloon unfolds completely but there is no elastic expansion of the balloon envelope.

The inversion depth B is calculated as follows: B>=N2 (>=represents greater/equal).

In mounting the balloon on the catheter shaft, the respective points for the inversion of each of the balloon shaft ends are the apex 8 of the distal, intrarectal balloon radius 9 and the apex 12 of the proximal, preanal balloon radius 13.

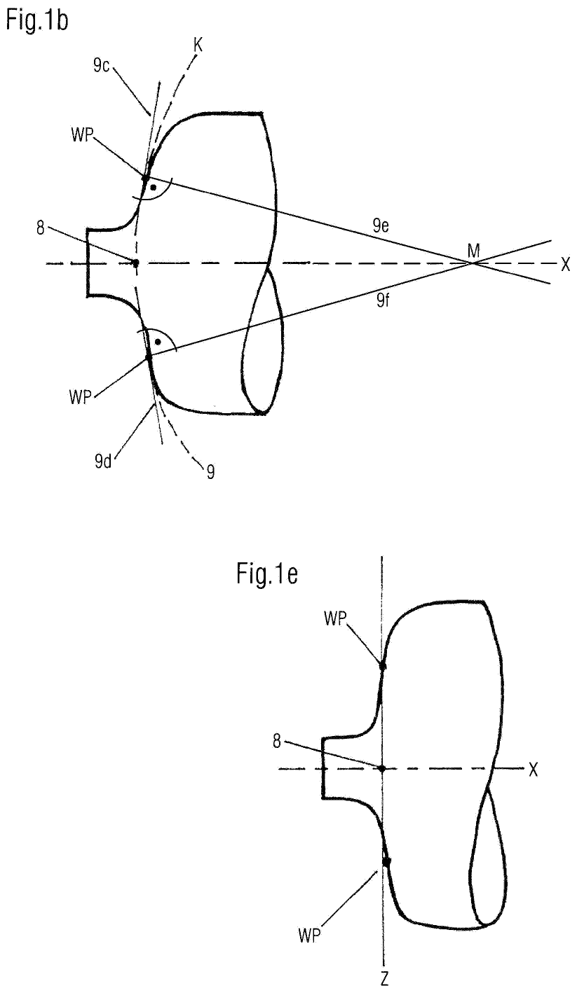

In the embodiment described in this figure, the distal fixation line 11 also corresponds to the distal end of the catheter shaft 1. The shaft terminates directly at the fixation line 11 and does not extend distally past this fixation line in the manner illustrated in FIG. 2.

FIG. 1b describes the geometric derivation of the apex of the distal balloon radius.

The forward balloon radius 9, frontally facing the intestinal lumen, of the intrarectal balloon segment 4 is illustrated as a broken line. It is constructed from the two inflection points 9a and 9b and the two inflection tangents 9c and 9d respectively belonging to these inflection points.

One of the two inflection points 9a or 9b is used to construct a straight line 9e or 9f that is normal to the respective inflection tangent 9c or 9d and intersects the corresponding inflection point 9a or 9b. The point of intersection of these straight lines 9e or 9f with the axis X of symmetry yields the center point of the circle K.

The circle K, and thus the forward balloon radius 9, results from the center point M of the circle and the inflection points 9a and 9b, which are on the circumference of the circle. In this derivation, apex 8 is obtained from the point of intersection of the circumference of the circle with the axis X of symmetry of the balloon.

For the description of the frontal apex 8 used below, the derivation of the farthest distally ranging point of the filled, unpressurized balloon envelope is performed, for purposes of simplification, using the point of intersection of the connecting line Z between the two inflection points 9a and 9b with the axis X of symmetry.

FIG. 1c shows the behavior of the catheter balloon that was inverted according to the invention in FIG. 1a as it unfolds freely and without pressurization, in the free, non-transanally placed state. The figure illustrates the counter-rolling movement of the two terminal balloon segments 4 and 5 made by possible by the specific inversion of the balloon ends 6 and 7 on the catheter shaft.

In a preferred, particularly thin-walled and soft-film-like implementation of the balloon body, the two segments move toward each other in response to the slightest, nearly ambient filling pressure and roll over the middle segment 3. When the two segments are in contact in the region of the transition points (15, 16) of the shoulder radii, the apex 8 of the radius 9 is flush or nearly flush with the distal fixation line 11 of the distal balloon shaft end 6 on the catheter shaft.

A configuration of this kind would correspond in situ to a clinical use situation in which the anal canal was maximally shortened in length. Even in this extreme case, it would thus be ensured by virtue of the described inversion rule that the free distal catheter shaft end, which here corresponds to the distal fixation line 11, does not extend into the intestinal lumen and even in the event of maximal lateral deflection of the catheter shaft in the rectum (tilting of the intrarectal portion of the shaft toward the bowel wall) does not come into contact with the wall of the intestine and pass beyond the distal balloon radius 9, as a maximum limit precluding irritation and lesions of the bowel wall.

FIG. 1d shows how the inversion of the balloon ends in relation to the distal catheter shaft ends described in FIG. 1a presents itself in cases where the anal canal is of normal or only slightly shortened length. In this more common clinical use situation, the forward fixation line 11, which here corresponds in turn to the distal free end of the catheter shaft, is shown to be deflected substantially into the interior of the intrarectal balloon 4. As the balloon is charged with pressure, the two terminal balloon segments 4 and 5 move counter to each other toward the anus and so conform to the particular anal situs. Given a suitably thin-walled implementation and soft-film-like character of the balloon envelope, the counter-rolling of the balloon segments commences even in response to a very low pressure force (filling pressure) that gives an impression of being nearly ambient to the environment.

The mere individual intra-abdominal pressure bearing against the transanally placed balloon or acting on the balloon is sufficient to produce a combined transanal sealing effect, comprised of radial sealing with respect to the anal canal and axially oriented sealing brought about by the counter-rolling movement of the terminal balloon enlargements at the inner and outer outlets of the anus. The sealing effect thus is not contingent on the balloon initially being filled beyond its volume and unfolding freely until the balloon envelope begins to expand. The balloon can behave in the described axial counter-rolling and radial unfolding fashion, even under partial filling of, for example, 70 to 90% of its volume when freely unfolded on the catheter shaft. This enables the balloon catheter to be placed in the anus in a nearly pressure-neutral and irritation-free manner.

In the presence of normal or slightly modified anal anatomy, any traumatizing effect of the tip of the catheter shaft on the bowel wall can thus be precluded, even under completely pressure-neutral charging with a filling medium, by the inversion of the shaft tip that occurs according to the invention.

FIG. 2 shows, by way of example, how sections of the catheter shaft in the form of a tip piece 18 that extend beyond the distal fixation line 11 in a distal prolongation directed toward the bowel are to be considered in determining the inversion depth of the balloon ends 6 and 7, in order to ensure atraumatic inversion, according to the invention, of the distal end of the catheter in the intrarectal balloon during transanal placement of the filled catheter balloon. The length C of the tip piece 18 is defined as the distance from the forward fixation line 11 to the forward apex 19 of the tip piece.

Length B is increased in comparison to FIG. 1a by the magnitude of length C or C/2.

The corresponding inversion depth B is preferably calculated taking into account a tip piece in the form: B>=A/2+C.

Alternatively hereto, the corresponding inversion depth B can, less preferably, be obtained considering a tip piece according to the relation: B>=A/2+C/2.

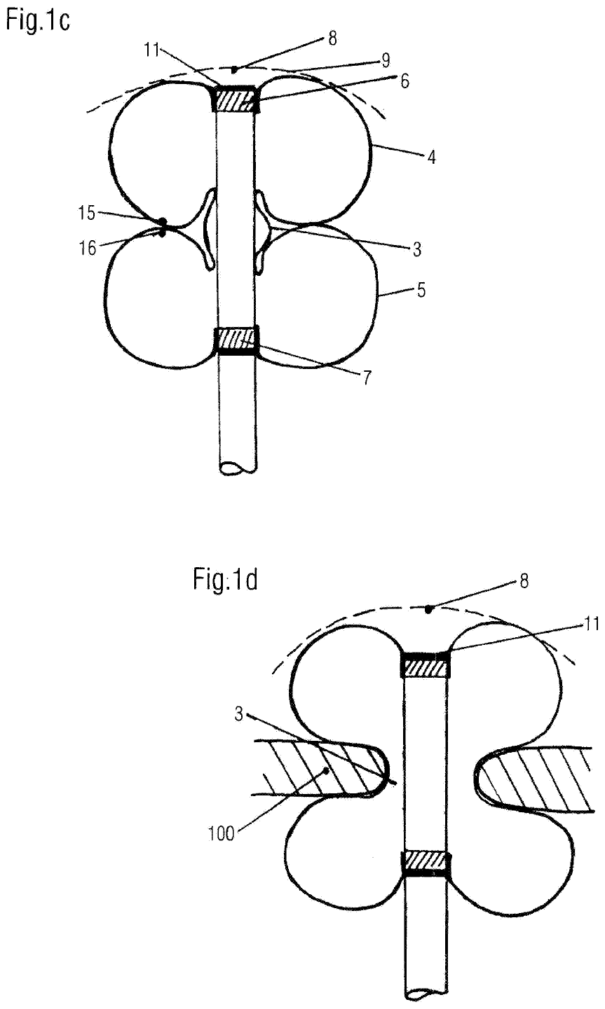

FIG. 3 illustrates another, alternative rule for establishing the inversion depth B of the balloon shaft ends 6 and 7 on the catheter shaft 1 in relation to the distal catheter shaft end 11, 19.

This rule particularly considers a possible axially oriented deflection of the catheter shaft inside the filled, transanally placed balloon. In the context of the inventively described inversion of the balloon shaft ends on the catheter shaft supporting the balloon, such deflection of the shaft in the longitudinal axis could cause the distal end of the catheter shaft to be deflected toward the bowel, thus creating a potential risk of perforation.

The maximum distal deflection W of the forward fixation line 11 is defined as a distance that emanates from the apex 8 of the radius 9, forming a distally directed prolongation of the longitudinal axis of the shaft, and extends to the apex 20 of a radius 21, said radius 21 being constructed over the largest diameter D of the intrarectal balloon segment 4.

If the catheter shaft has a tip piece 18 that extends beyond line 11, the maximum deflection path W should be correspondingly selected so that upon maximum deflection W of the shaft, the tip 19 of the tip piece does not extend past radius 21.

The radius 21 defined by the largest diameter D in the intrarectal balloon segment constitutes a fundamentally relevant boundary line for distal portions of the catheter shaft. In the event of lateral tilting of the shaft longitudinal axis of the transanally placed catheter shaft, the fact that the maximum deflection W is referred to the largest balloon diameter D ensures that the catheter shaft tip (11, 19) still moves within the pivot radius 21 of balloon segment 4, thus preventing relatively well any potential traumatizing contact of the tip with the bowel wall adjacent the balloon.

In determining the inversion depth B, the particular ratio of the distance W to the radius 21 or of the diameter D on which it is based is preserved and the inversion depth B is adjusted accordingly as necessary.

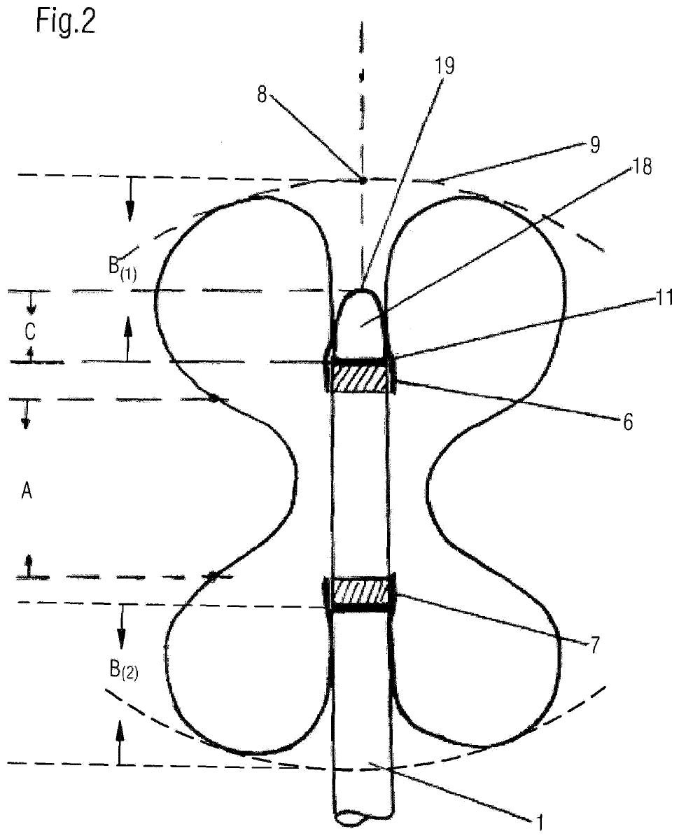

FIG. 4 shows the catheter balloon 2 in its emptied form, in which it lies against the catheter shaft ready for insertion in the anus. The envelope segments of the intrarectal balloon 4 and of the middle segment 3 cling to the shaft in folds.

In this state, the two envelope portions preferably come to lie approximately at the height of the line segment between the fixation points of the balloon ends 6 and 7 on the surface of the shaft. The envelope of the preanal balloon segment 5, by contrast, is preferably smoothed out in the proximal direction and protrudes past the fingers gripping the catheter for insertion, the preferred gripping point being located just proximal of the proximal fixation line 14. The gripping point 22 is preferably implemented as a depression-like receiving surface, one such preferably being provided on each of the opposite, 180°-apart shaft surfaces.

The evacuated balloon being fixed in such fashion, the user grips with his fingers under the envelope of segment 5, which envelope is smoothed out in a proximal direction, and guides the catheter into the rectum until the gripping fingers abut the external anus. A defined insertion depth is thereby ensured. It is further ensured that the intrarectal balloon section 4 is inserted into the rectal cavity, while the proximal balloon portion 5 comes to lie outside the anus (preanally). Reliable transanal positioning of the catheter therefore occurs as the waisted balloon is filled.

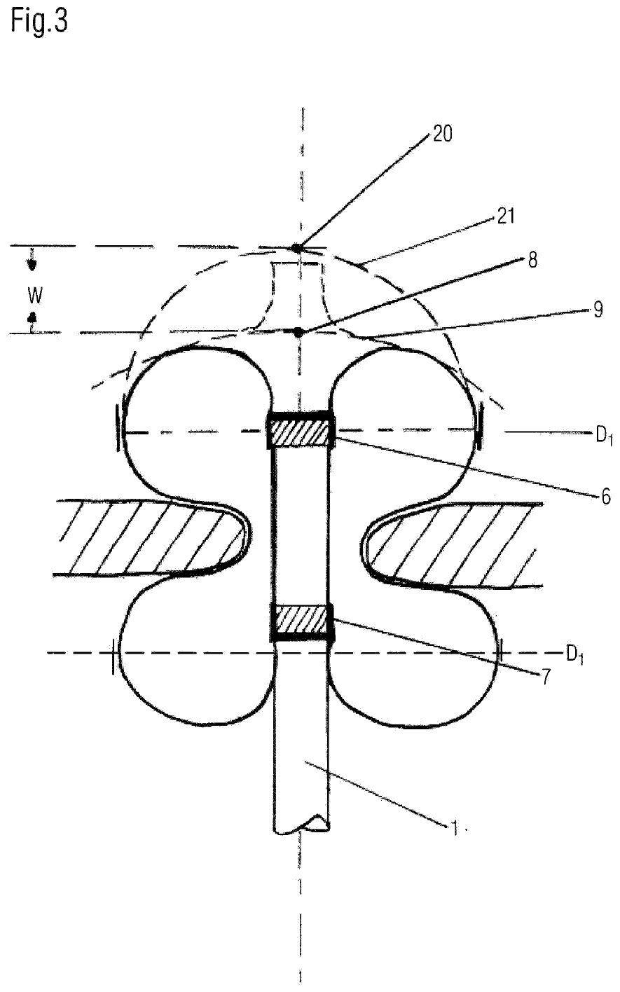

FIG. 5 shows a preferred implementation of the shaft body 1 that furnishes some degree of automatic positioning and securing of the shaft in the anus even when the catheter balloon has not yet been filled. The shaft 1 has for this purpose a likewise waisted shape, which is correspondingly tapered in the transanal region 23 and in effect locks the catheter shaft in a transanal position in this region after insertion. The catheter shaft preferably also has a distally terminal, funnel-like, atraumatically shaped opening 24 that connects to the duct 25 supplying the medium.

In a particularly large-volume implementation of the intrarectal balloon segment or a longitudinal expansion of the balloon segment that reaches far into the rectum, the balloon segment, in the non-air-filled, ready-to-use state, can optionally be stuffed or packed partially into the opening 24. It then slips out of the opening as the inserted catheter is filled.

Given suitable implementation of the shaft material, the waist 23 can also predefine a certain kinkability of the shaft body, thus improving its atraumatic properties.

The filling of the balloon takes place through a separate duct 26 integral to the shaft.

To prevent backflow of irrigation fluid, the inflow catheter can is [sic] equipped with a non-return valve in the region of the fluid-conveying duct 25.

The valve can preferably consist of a thin-walled tube element having a wall thickness of few, preferably, 5 to 15 micrometers and the diameter of the duct 25, the distal end of the tube lying freely in the duct 25 over a length of approximately 5 to 10 mm and its proximal end being connected sealingly to the inner wall of the duct 25. As the medium flows through the duct in the direction of the tip, the tube element opens and allows the medium to flow freely. In response to flow in the reverse direction, the tube element collapses and closes up sealingly, preventing an effective backflow.

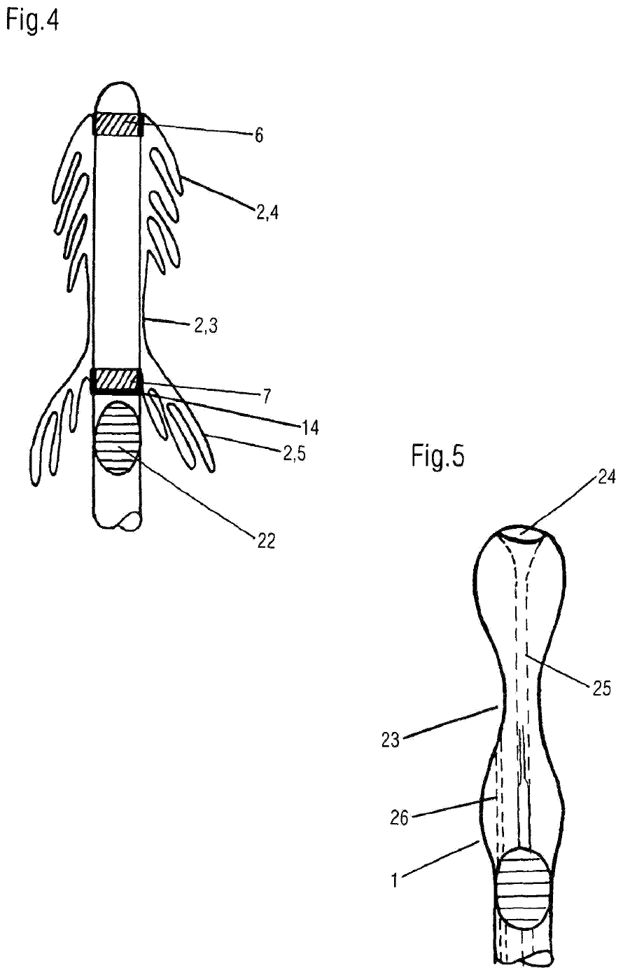

FIG. 6 schematically illustrates a pump manometer 27 equipped with a scale 28 suitable for the filling of the inventive catheter balloon in multiple steps or with a sequential increase in filling pressure. The scale preferably displays, on the one hand, an initial, low pressure range 29 (app. 10-25 mbar), which is set by the user after inserting the catheter and before introducing the infusion fluid, and which, given the inventive preshaping of the balloon envelope and its fixation on the shaft, is sufficient in most cases to ensure transanal anchoring and sealing of the catheter without a direct reflex-triggering effect on the bowel wall.

When the user then initiates an increase in the filling pressure in the balloon into range 30 (30-60 mbar, with increasing corresponding expansion of the rectal portions of the bowel wall) or into range 37 (60-120 mbar, with increasing additional expansion of the anal sphincter), the user is ultimately able to generate a trigger stimulus of largely reproducible intensity in order to trigger a defecation reflex. The user thus has the advantage of being able to avoid an evacuation reflex in the presence of an initially low balloon pressure merely having an anchoring and sealing action, and thus of being able to retain the infusion fluid in the bowel long enough for his individual needs, resulting in better dissolution or suspension of stool in the fluid. On the other hand, by intentionally causing an increase in pressure in the balloon, he can generate an intense, relatively prompt-acting reflex-triggering stimulus, which can, if necessary, exceed in intensity the triggering effect of a column of colorectal fluid.

The catheter balloon is preferably filled with air, through a fill line integrated into the shaft wall of the catheter body 1.

In addition to pressure-controlled filling of the balloon using a pump manometer, as illustrated in FIG. 6, volume-controlled filling can be achieved with the use of an additional syringe element that specifies the preferred fill volume by means of a suitable marking on the syringe body. The filling of the balloon is preferably partial, in the form of one-step filling. In the ideal case, therefore, in loose, unexpanded form, the balloon lies against the structures of the bowel and the anus. The balloon envelope thus absorbs the forces acting on the balloon in the rectum, the anus and the preanal region and brings the balloon into the configuration in which it performs the anchoring and sealing function. The respective physiologically acting forces are absorbed by the catheter balloon and thus enable the balloon to be placed anorectally in as pressure-neutral a manner as possible, largely precluding undesired and premature triggering effects.

A volume-controlled filling of the balloon can also be a two-step process, in which incomplete filling is first performed and the balloon is then, in the second filling step, filled with a volume that has a triggering effect. The pressures developed in the transanal balloon in response to the particular volume should preferably fall within the pressure ranges (29, 30) described in FIG. 6, and are to be determined by and for the user on an individual basis, as in the case of pressure-controlled filling.

To limit the filling pressure or prevent critically high balloon filling pressures, in the case of both manometer- and syringe-actuated filling a pressure limiting valve 31 can be interposed between the filling element and the catheter, to prevent, for example, balloon filling pressures over 120 mbar.

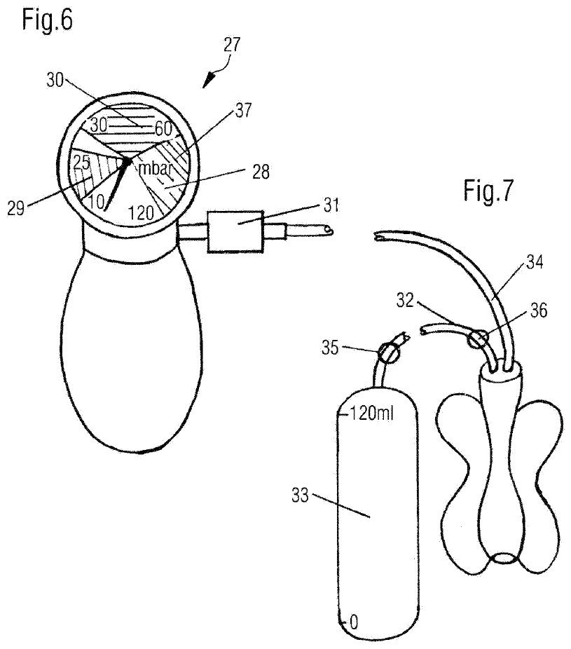

FIG. 7 shows a preferred embodiment of an inflow catheter, which transitions at the proximal end to two feed conduits that are preferably fixedly connected to the shaft, feed conduit 32 being fixedly connected to a preferably bag-like container 33 containing infusion medium, and thus constituting a ready-to-use unit comprised of catheter and medium.

The volume of the container 33 must be dimensioned in this case to accommodate approximately 80 to 120 ml of irrigation solution. The relatively small quantity is squeezed out manually by the user and thus introduced into the rectum by repeated squeezing. To make it possible to grip the container even with impaired hand motor function, the container is preferably shaped as cylindrical, with a diameter of approximately 4-6 cm.

Connection 32 is preferably equipped with a seal 35 that can be broken by bending and that releases the irrigation solution. It is also advantageous to provide a non-return valve 36, which is integrated into the fluid-conveying arm of the ready-to-use device and which permits the directed emptying of the container without backflow.

Another feed conduit 34 can be connected directly to a pump manometer 27 or a filling syringe.

The balloon element 2 consists of a thin-walled soft film in the wall thickness range of 5 to 100 micrometers. Films in the thickness range of 5 to 40 micrometers are advantageous. Wall thicknesses of 5 to 15 micrometers, on the other hand, are particularly preferable.

The use of only slightly volume-expandable materials, such as, for example, polyurethane (PUR), for example of the specification Pellethane 2363 80A to 90A, Dow Chemical Corp., is preferred, since these materials have good dimensional stability in the lowest wall thickness range, including as balloon films, in the pressure range of approximately 10 to 120 mbar.

Such thin-walled PUR balloon films formed into complex shape can preferably be produced by hot molding from previously extruded raw tubing material, which, with suitable stretching of the tube blank before tempering, permits a polymer orientation and lends the shaped balloon films exceptional mechanical strength.

It is also conceivable to use polyurethanes of low Shore hardness, for example in the range of 60 to 75 A, to impart a volume-expandable behavior, with a relative loss of dimensional stability, to the catheter balloon in the wall thickness range of less than 40 μm, and preferably less than 15 μm, in the typical filling pressure range during use of 10 to 120 μm.

Alternatively, for example non-volume-expandable materials can also be used, such as polyethylene, PVC or mixtures of the aforesaid materials with polyurethane.

Balloon films according to the invention can also be shaped directly from the extruded, still soft, largely amorphous tube molding compound (in-line molding), in which case the achievable strengths of the films are much lower than those of pre-extruded tubes and the attainable wall thicknesses are much higher than in the case of forming from pre-extruded material.

Dipping processes using liquid PVC or PUR materials can also be contemplated for production.

The welding together of single layers of film to form balloon bodies is also conceivable.

The connection of the balloon to the shaft body is effected by gluing, by thermal methods, or alternatively by shrinking the balloon ends onto the shaft body.

In the freely unfolded, unpressurized state, the balloon should preferably have a diameter of approximately 30-60 mm in the intrarectal section 4, approximately 10 to 30 mm in the middle, waisted region 3, and approximately 30-50 mm in the preanal region 5. The middle segment 3 should have a length of approximately 20-40 mm and each of the terminal segments 4 and 5 a length of approximately 20-40 mm.

If, as a special measure for sequential filling, the catheter balloon is provided with optional intrarectal balloon triggering, the intrarectal balloon section 4 in the freely unfolded, unpressurized state preferably has a diameter of approximately 40-80 mm and a length of preferably 30-60 mm.

In addition to the transanal use of the inflow catheter, the embodiments according to the invention can also be used for perforation-safe placement of a transanally inserted drainage tube for, among other purposes, the ongoing drainage of stool from a patient's intestine. Further, the described catheter technique may also be contemplated for use in surgically created stomata/openings or other natural body openings.

1. A method for intermittently triggering a coordinated defecation reflex in the rectum or colon of a user with an individual anatomy of the rectum or colon and a current reflex status, comprising the following steps:
providing a catheter, the catheter comprising:
a catheter shaft, and
a catheter balloon surrounding the catheter shaft and being fixed to it, wherein the catheter balloon comprises a balloon envelope:
which has a tubular or an annular geometry with two ends which are fixed to the catheter shaft at two fixation points in a fluid-tight manner in order to encompass a fluid-tight fillable compartment, wherein the distal fixation point of the catheter balloon is near the distal end of the catheter shaft, and
which surrounds the catheter shaft between the two ends of the balloon in a freemoving manner, as the balloon envelope is not attached or fixed to the catheter shaft at any third location between the two fixation points of the ends of the balloon envelope at the catheter shaft, and
which, during manufacturing of the catheter balloon, is preshaped to a reference dimension (V_{REF}) of a balloon filling volume (V_{BF}) as a maximum filling volume of the compartment in the balloon, which is achievable by inflation, but without any stretching of the balloon envelope, and which reference dimension (V_{REF}) of the balloon filling volume (V_{BF}) corresponds to a dimension of the rectum or colon of the user, and
which is manufactured from a material which, although being flexible, is non-stretchable or only slightly stretchable so that, at or above the reference dimension (V_{REF}), the ballon filling volume (V_{BF}) of the catheter balloon instantly assumes or asymptotically approximates an unvarying dimension limit (V_{UDL}) at a pressure in the range of 10 to 120 mbar applied to the interior of the balloon:
V_{UDL}>V_{REF};
in a first step, inserting the catheter in the rectum or colon of the user and providing an initial fill volume (V_{IF,1}) or an initial filling pressure (p_{IF,1}) of air or another fluid in the catheter balloon in order to inflate the catheter balloon below the reference dimension (V_{REF}) of the balloon filling volume (V_{BF}) thereof in a pressure-neutral manner, i.e. wherein the initial filling pressure (p_{IF,1}) inside the catheter balloon is equal to a physiologically acting intra-rectal acting pressure (p_{IR}) that is prevailing in the rectum of the user, producing a sealing and anchoring effect without any stretching of the envelope of the catheter balloon and causing neither a trans-mural pressure (p_{TM,1}) beyond the physiologically intra-rectally acting pressure (p_{IF}) nor a reflex triggering expansion of portions of the lateral rectal wall exposed to the catheter balloon, according to the following formulas:
p_{IF,1}=p_{TM,1}=p_{IR},
and wherein the initial fill volume (V_{IF,1}) of the catheter balloon is below the reference dimension of the balloon filling volume (V_{REF}):
V_{IF,2}<V_{REF};
so that the envelope of the balloon is not stretched; and
in a second step, increasing such initial fill volume (V_{IF,1}) or such initial filling pressure (p_{IF,1}) to an increased fill volume (p_{IF,2}) or an increased filling pressure (p_{IF,2}) by pumping, squeezing or injecting air or another fluid into the catheter balloon by the user until there occurs a gradual, user-controllable expansion of portions of the lateral rectal wall exposed to the catheter balloon in such a way that the degree of filling of the balloon is best adapted to the individual anatomy, allowing for an even distribution of the trans-mural pressure (p_{TM,2}) exerted by the balloon onto the exposed rectal structures, gradually increasing from the physiologically acting intra-rectal pressure (p_{IR}) to a force level suited for triggering a coordinated physiological defecation reflex of the individual user, according to the following formula:
p_{IF,2}=p_{TM,2}>p_{IR},
and wherein the increased fill volume (V_{IF,2}) of the catheter balloon is still below the reference dimension of the balloon filling volume (V_{REF}):
V_{IF,2}<V_{REF};
so that the envelope of the balloon is not stretched.

2. The method according to claim 1, wherein if placed in situ, the proximal fixation point of the catheter balloon to the catheter shaft is either suited upstream the anal canal for an intra-rectal unfolding the balloon only, or is suited in the anal canal for an expansion of a rectal balloon segment into the anal canal, or is situated beyond the anal canal for an expansion of the rectal balloon segment throughout the anal canal, forming a pre-anal balloon segment.

3. The method according to claim 1, wherein the reference dimension (V_{REF}) of the balloon filling volume (V_{BF}) is pre-shaped to a volume of 30 ml to 150 ml.

4. The method according to claim 1, wherein, if the balloon filling volume (V_{BF}) is below the preshaped reference-dimension (V_{REF}), the envelope of the catheter balloon is not free of wrinkles.

5. The method according to claim 1, wherein, in a reference state where the ballon filling volume (V_{BF}) reaches the preshaped reference-dimension (V_{REF}), the balloon adopts a geometrical shape that is unfolding/expanding in a radial direction more than in an axial direction of the catheter shaft.

6. The method according to claim 1, wherein the reference dimension (V_{REF}) of the balloon filling volume (V_{BF}) is designed in such a way, that, if the balloon is inflated up to this reference dimension (V_{REF}) or beyond, it adopts a discoidal geometrical shape.

7. The method according to claim 6, wherein the discoidal shape is characterized in that a distance between a peripheral-most extension of the toroidal balloon and the lateral surface of the catheter shaft is at least equal to the distance between the two fixation points of the envelope of the catheter balloon to the catheter shaft.

8. The method according to claim 1, wherein the reference dimension (V_{REF}) of the balloon filling volume (V_{BF}) is designed in such way, that, if the balloon is inflated up to this reference dimension (V_{REF}) or beyond, it does not extend beyond the distal-most tip of the shaft.

9. The method according to claim 1, wherein the unvarying dimension limit (V_{UDL}) of the balloon filling volume (V_{BF}) is designed in such way, that, if the balloon is inflated up to this unvarying dimension limit (V_{UDL}), it does not extend beyond the distal-most tip of the shaft.

10. The method according to claim 1, wherein the unvarying dimension limit (V_{UDL}) of the balloon filing volume (V_{BF}) is within a range of 100% to 150% of the reference dimension (V_{REF}) of the balloon filling volume (_{VBF}).

11. The method according to claim 1, wherein, if the balloon filing volume (V_{BF}) is further increased to a further increased balloon filing volume (V_{IF,3}) above the reference dimension (V_{REF}):
V_{IF,3}>V_{REF},
then the balloon filling pressure (p_{IF,3}) is at a higher level than a regarding trans-mural pressure (p_{TM,3}):
p_{IF,3}>p_{TM,3}>p_{IR}.

12. The method according to claim 1, wherein the material of the catheter balloon is polyurethane (PUR).

13. The method according to claim 1, wherein the material of the catheter balloon is selected from the group consisting of polyethylene, PVC and mixtures of polyethylene or PVC with polyurethane.

14. The method according to claim 1, wherein, in the first step, the initial fill volume (V_{IF,1}) is between 10 ml and 20 ml.

15. The method according to claim 1, wherein, in the first step, the initial filling pressure (p_{IF,1}) is between 10 mbar and 30 mbar, or between 10 mbar and 25 mbar.

16. The method according to claim 1, wherein, in the second step, the increased fill volume (V_{IF,2}) is below the reference dimension (V_{REF}) of the balloon filling volume (V_{BF}), so that the envelope of the balloon is not wrinkle-free.

17. The method according to claim 1, wherein, in the second step, the increased fill volume (V_{IF,2}) is between 30 ml and 150 ml.

18. The method according to claim 1, wherein, in the second step, the increased filling pressure (p_{IF,2}) is between 30 mbar and 60 mbar.

19. The method according to claim 1, wherein a safety range is provided at or above the reference dimension (V_{REF}) of the balloon filling volume (V_{BF}), to ensure that the envelope of the balloon will not substantially enlarge beyond such reference dimension (V_{REF}) of the balloon filling volume (V_{BF}).

20. The method according to claim 1, wherein during the second step, the catheter balloon remains in a pressurized state for about 1 minute to 5 minutes, until a coordinated physiological defecation reflex is triggered, wherein an uncoordinated and only partially productive defecation reflex is avoided.

21. The method according to claim 1, wherein, in the first and or second step, the air or fluid is pumped, squeezed or injected manually, especially by the user himself.

22. The method according to claim 1, wherein, between the first and second step, a liquid irrigation inflow fluid is introduced directly into the rectum of the user.

23. The method according to claim 22, wherein the liquid irrigation inflow fluid is introduced into the rectum of the user via a central lumen of the catheter shaft, which is open at a distal tip of the catheter shaft.

24. The method according to claim 22, wherein the liquid irrigation inflow fluid is introduced into the rectum of the user by squeezing it out of a container.

25. The method according to claim 24, wherein the liquid irrigation inflow fluid is delivered as a ready-to-use solution through a fixed feed conduit to the catheter, as a disposable product.

26. The method according to claim 22, wherein the liquid irrigation inflow fluid is introduced into the rectum of the user in a volume (V_{LIIF}) of up to 120 ml, or in a volume (V_{LIIF}) of 80 to 120 ml, or in a volume (V_{LIIF}) of even less than 80 ml.

27. The method according to claim 1, wherein a volume (V_{LIIF}) of a liquid irrigation inflow fluid introduced directly into the rectum of the user outside of the catheter balloon acts together with the balloon filling volume (V_{BF}) as a pneumatic expansion stimulus that can be controlled by the user.

28. The method according to claim 27, wherein volume (V_{LIIF}) of a liquid irrigation inflow fluid fills the rectum or colon of the user above the balloon filling volume (V_{BF}) which is located near by or adjacent to the anus of the user.

29. The method according to claim 1, wherein the catheter further comprises a filling conduit for charging the catheter balloon with filling pressure.

30. The method according to claim 1, wherein a reusable filling device is provided for filling the balloon in the first and/or second step.

31. The method according to claim 30, wherein the reusable filling device is a hand-operated pump balloon connectable to the catheter balloon via a coupling.

32. The method according to claim 30, wherein the reusable filling device is a pump balloon with a pressure-indicating manometer.

33. The method according to claim 32, wherein the pressure-indicating manometer has a scale which displays one or more pressure ranges, recommended for various filling steps when applying the device.

34. The method according to claim 1, wherein the filling operation of the balloon with the initial or increased fill volume is conducted by controlling a balloon filling volume (V_{BF}).

35. The method according to claim 34, wherein the filling operation is conducted by use of a syringe element.

36. The method according to claim 35, wherein a syringe body is used which comprises a suitable marking that specifies a preferred balloon filling volume (V_{BF}).

37. The method according to claim 1, wherein the catheter shaft is equipped in a proximal, preanal region with gripping depressions, to accommodate fingers gripping the catheter during insertion and to ensure a safe catheter insertion.

38. The method according to claim 37, wherein the gripping depressions are disposed on the catheter shaft in direct proximal adjacency to a rear fixation line of the catheter balloon.

39. The method according to claim 1, wherein the balloon filling pressure (p_{BF}) is limited by a pressure limiting valve which is interposed between a filling element and the balloon of the catheter.

40. The method according to claim 1, wherein, upon a further increase of the balloon filling pressure (p_{BF}) prevailing in the catheter balloon to approximately 60 to 120 mbar, the anal canal of the user is also caused to expand.

41. The method according to claim 1, wherein, as soon as a coordinated physiological defecation reflex is triggered, the catheter balloon is deflated and the catheter is extracted from the rectum.

42. A device for intermittently triggering a coordinated defecation reflex in the rectum or colon of a user with an individual anatomy of the rectum or colon and a current reflex status, comprising a catheter, the catheter comprising:
a catheter shaft, and
a catheter balloon surrounding said catheter shaft and being fixed to it, wherein the catheter balloon comprises a balloon envelope:
which has has a tubular or an annular geometry with two ends which are fixed to the catheter shaft at two fixation points in a fluid-tight manner in order to encompass a fluid-tight fillable compartment, wherein the distal fixation point of the catheter balloon is near the distal end of the catheter shaft, and
which surrounds the catheter shaft between the two ends of the balloon in a freemoving manner, as the balloon envelope is not attached or fixed to the catheter shaft at any third location between the two fixation points of the ends of the balloon envelope at the catheter shaft, and
which, during manufacturing of the catheter balloon, is preshaped to a reference dimension of a balloon filling volume as a maximum filling volume of the compartment in the balloon, which is achievable by inflation, but without any stretching of the balloon envelope, and which reference dimension of the balloon filling volume corresponds to a dimension of the rectum or colon of the user,
which is laid in wrinkles if the balloon is inflated to a reference dimension of the filling volume below the preshaped reference dimension of the filling volume, and
which is manufactured from a material which, although being flexible, is non-stretchable or only slightly stretchable so that, at or above the reference dimension, the ballon filling volume of the catheter balloon instantly assumes or asymptotically approximates an unvarying dimension limit at a pressure in the range of 10 to 120 mbar applied to the interior of the balloon;
a manually operable pump, optionally with an integrated manometer for a pressure-controlled filling of the catheter balloon by the user, or a manually operable syringe element for a volume-controlled filling of the catheter balloon by the user:
by providing an initial fill volume or an initial filling pressure to the catheter balloon in order to inflate it to a balloon filling volume below the reference dimension, which is sufficient to achieve an anchoring effect of the catheter balloon in the rectum or colon in a pressure-neutral manner, but without causing a reflex triggering expansion of portions of the bowel wall adjacent to the catheter balloon, and
by pumping, squeezing or inserting air or another fluid into the catheter balloon in order to to increase said initial fill volume or said initial filling pressure to an increased fill volume or to an increased filling pressure until there occurs a gradual, user-controllable expansion of portions of the bowel wall adjacent to the catheter balloon, thus triggering a coordinated defecation reflex.

43. A method for intermittently triggering a coordinated defecation reflex in the rectum or colon of a user with an individual anatomy of the rectum or colon and a current reflex status, comprising:
providing a catheter, the catheter comprising:
a catheter shaft, and
a catheter balloon surrounding the catheter shaft and being fixed to it, wherein the catheter balloon comprises a balloon envelope:
which has a tubular or an annular geometry with two ends which are fixed to the catheter shaft at two fixation points in a fluid-tight manner in order to encompass a fluid-tight fillable compartment, wherein the distal fixation point of the catheter balloon is near the distal end of the catheter shaft, and
which surrounds the catheter shaft between the two ends of the balloon in a freemoving manner, as the balloon envelope is not attached or fixed to the catheter shaft at any third location between the two fixation points of the ends of the balloon envelope at the catheter shaft, and
which, during manufacturing of the catheter balloon, is preshaped to a reference dimension of a balloon filling volume as a maximum filling volume of the compartment in the balloon, which is achievable by inflation, but without any stretching of the balloon envelope, and which reference dimension of the balloon filling volume corresponds to a dimension of the rectum or colon of the user, and
which is manufactured from a material which, although being flexible, is non-stretchable or only slightly stretchable so that, at or above the reference dimension, the ballon filling volume of the catheter balloon instantly assumes or asymptotically approximates an unvarying dimension limit at a pressure in the range of 60 to 120 mbar applied to the interior of the balloon;
inserting the catheter in the rectum or colon of the user,
providing an initial fill volume or an initial filling pressure of air in the catheter balloon in order to inflate the catheter balloon to a catheter balloon volume below its reference dimension without any stretching of the envelope of the catheter balloon, thereby achieving an anchoring effect of the catheter balloon in the rectum or colon of the user, and
expanding portions of the bowel wall adjacent to the catheter balloon in order to trigger a coordinated defecation reflex by raising the initial filling pressure to an increased filling pressure in the range of 30 to 60 mbar, by additionally pumping, squeezing or inserting air or another fluid into the catheter balloon by the user until there occurs a gradual, user-controllable expansion of portions of the bowel wall adjacent to the catheter balloon in such a way that the degree of filling of the balloon is best adapted to the individual anatomy and current reflex status of the user in order to trigger the defecation reflex.

44. (canceled)
